(19) United States
(12) Patent Application Publication
(10) Pub. No.: US 2024/0307429 A1
(43) Pub. Date: Sep. 19, 2024

(54) ANTI-VIRAL PHOSPHORAMIDATES AND USES THEREOF

(71) Applicants: Carston R. Wagner, St. Paul, MN (US); Luciana Jesus Costa, Minneapolis, MN (US); Ramario Mattos, Minneapolis, MN (US)

(72) Inventors: Carston R. Wagner, St. Paul, MN (US); Luciana Jesus Costa, Minneapolis, MN (US); Ramario Mattos, Minneapolis, MN (US)

(21) Appl. No.: 18/605,377

(22) Filed: Mar. 14, 2024

Related U.S. Application Data
(60) Provisional application No. 63/490,133, filed on Mar. 14, 2023.

(51) Int. Cl. A61K 31/708; A61P 31/14

(52) U.S. Cl. CPC A61K 31/708; A61P 31/14

(57) ABSTRACT

Described herein are compounds, and pharmaceutically acceptable salts thereof, as well as methods of using those compounds to, among other things, treat severe acute respiratory syndrome.

### CROSS-REFERENCE TO RELATED APPLICATIONS

This application claims the benefit of U.S. Provisional Appl. No. 63/490, 133, filed Mar. 14, 2023, which is incorporated by reference as if fully set forth herein.

### STATEMENT OF U.S. GOVERNMENT SUPPORT

This invention was made with government support under AI146049 awarded by the National Institutes of Health. The government has certain rights in the invention.

### BACKGROUND

Coronaviruses (CoVs) are enveloped viruses with a positive-sense, single-stranded RNA and are associated with various natural hosts. CoVs are divided into alpha, beta, gamma, and delta groups, and the beta group is further composed of A, B, C, and D subgroups. Among them, six CoVs can infect humans (HCoVs), including HCoV-229E (229E) and HCoV-NL63 (NL63) in the alpha group, HCoV-OC43 (OC43) and HCoV-HKU1 (HKU1) in beta subgroup A, severe acute respiratory syndrome CoV (SARS-CoV) in beta subgroup B, and Middle East respiratory syndrome CoV (MERS-CoV) in beta subgroup C.

In this century, SARS-CoV and MERS-CoV have emerged in the human population and caused severe pulmonary disease with alarmingly high fatality rates. In 2002, SARS-CoV infections first appeared in China and then quickly spread as a global pandemic to more than 30 countries with 8,273 infections and 775 deaths (nearly 10% mortality). In 2012, MERS-CoV emerged in Saudi Arabia and spread throughout the Middle East. In 2015, the second outbreak of MERS-CoV occurred in South Korea, causing super-spreading events with third-and fourth-generation cases of infection. The World Health Organization has reported 2,229 laboratory-confirmed cases of MERS-CoV infection, including 791 deaths (about 35% case fatality) in 27 countries as of August 2018 (the worldwide web at who[dot]int/emergencies/mers-cov/en/). Meanwhile, the remaining common HCoVs, such as 229E, OC43, and NL63, usually infect the human upper respiratory tract and cause the common cold, but they also are responsible for severe and even fatal diseases in children, the elderly, and immunocompromised patients. These scenarios suggest that those common HCoVs might also pose a lethal threat to humans. Note that HCoVs are rapidly evolving. OC43 isolates with novel genomes are being continuously identified.

The ongoing outbreak of coronavirus disease (COVID-19) originated in China in December 2019 and became a global pandemic by March 2020. COVID-19 is caused by a novel coronavirus, severe acute respiratory syndrome—coronavirus 2 (SARS-CoV-2). Two other coronaviruses have caused worldwide outbreaks in the past two decades, namely SARS-CoV (2002-2003) and Middle East respiratory syndrome coronavirus (MERS-CoV) (2012-present). There is currently no treatment for COVID-19. Therefore, the development of a drug that could inhibit SARS-CoV-2 would address an urgent unmet medical need.

Influenza, like SARS-CoV-2, is a serious public health problem with a high incidence in the human population resulting in regular large-scale morbidity and mortality. It is a highly contagious airborne disease that causes an acute febrile illness. Systemic symptoms vary in severity from mild fatigue to respiratory failure and death.

In the US, annual influenza epidemics lead to approximately 30 million outpatient visits, resulting in medical costs of $10 billion annually. Lost earnings due to illness and loss of life represent a cost of over $15 billion annually and the total US economic burden of annual influenza epidemics amounts to over $85 billion.

Although vaccination remains the main prophylactic strategy for controlling influenza infection, as well as for blunting the SARS-CoV-2 symptoms, to bridge the period before a new vaccine becomes available and to treat the severe influenza cases, as well as to counter the problem of viral resistance, a wider choice of anti-influenza drugs is required. Development of new influenza antivirals has therefore again become a high priority and an unmet medical need.

### DESCRIPTION OF THE FIGURES

The drawings illustrate generally, by way of example, but not by way of limitation, various embodiments discussed in the present document.

FIG. 1A is a bar graph showing an assessment of cellular toxicity of compound “4Ei-10” and “4Ei-11” using Vero E6 cells, wherein 4Ei-10 and 4Ei-11 have the formulae:

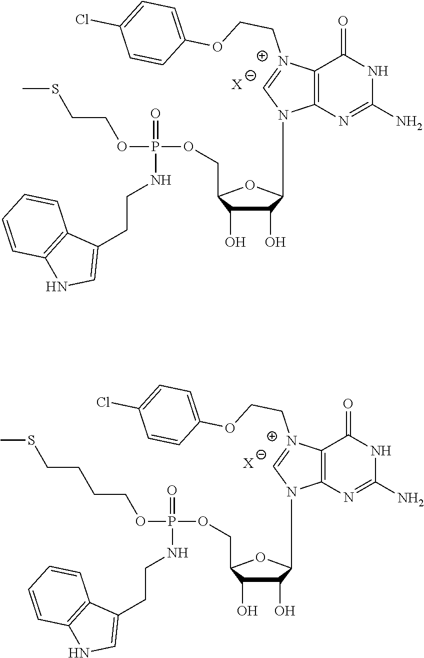

respectively.

FIG. 1B is a bar graph showing the effect of titrating SARS-CoV-2 (A2 lineage) infectious particles with compound 4Ei-10 and 4Ei-11 in culture supernatant by plaque assay. Vero E6 cells were pretreated for 4 hours with each compound at different concentrations (from 3 μM to 100 μM) followed by infection (MOI 0.1).

FIG. 2A is a bar graph showing an assessment of cellular toxicity of compound 4Ei-10 and 4Ei-11 using Calu-3 cells.

FIG. 2B-2E are bar graphs showing the effect of titrating SARS-CoV-2 (A2 lineage) infectious particles with compound 4Ei-10 and 4Ei-11 in culture supernatant by plaque assay. Calu-3 cells were pretreated for 4 hours with each compound at different concentrations (from 0.09 μM to 3 82 M) followed by infection (MOI 0.1).

FIG. 3A is a confocal microscope visualization of SARS-CoV-2 Spike protein and dsRNA in Calu-3 cells treated with 4Ei-10. Calu-3 were pretreated with each compound at 0.38 μM for 4 hours followed by infection with SARS-CoV-2 (Lineage A) at an MOI of 0.1.

FIG. 3B is bar graphs showing the quantification of the images acquired by confocal microscopy. ***p<0.001; ****p<0.0001 by One-way ANOVA.

FIG. 4A is a confocal microscope visualization of SARS-CoV-2 Spike protein and dsRNA in Calu-3 cells treated with 4Ei-11. Cells were pretreated with each compound at 0.38 μM for 4 hours followed by infection with SARS-CoV-2 (Lineage A) at an MOI of 0.1.

FIG. 4B is bar graphs showing Quantification of the images acquired by confocal microscopy. *p<0.05; ***p<0.001; ****p<0.0001 by One-way ANOVA.

FIG. 5A is a bar graph showing Antiviral activity of 4Ei-10 and 4Ei-11 against SARS-CoV-2 (Omicron VOC). Titration of SARS-CoV-2 infectious particles in culture supernatant by plaque assay. Cells were pretreated for 4 hours with each compound at 3 μM to 100 μM followed by infection (MOI 0.1).

FIG. 5B is a bar graph showing the percentage of inhibition of the amount of viral dsRNA produced. Cells were pretreated for 4 hours with each compound at 3 μM to 100 μM followed by infection (MOI 0.1).

### DESCRIPTION

Reference will now be made in detail to certain examples of the disclosed subject matter. While the disclosed subject matter will be described in conjunction with the enumerated claims, it will be understood that the exemplified subject matter is not intended to limit the claims to the disclosed subject matter.

The instant disclosure generally relates to compounds for treating a severe acute respiratory syndrome. The disclosure also relates to compounds for treating an influenza viral infection (e.g., influenza A, B and C viruses and subtypes, of the influenza A virus, including H1N1, H2N2, H3N2, and H5N1 subtypes, with influenza A viruses being the most common form that can spread in mammals and birds). These compounds have the general formula (I):

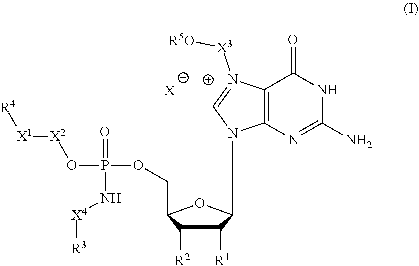

or a pharmaceutically acceptable salt thereof;
wherein:
X⁻ is a counterion, such as chloride and the like;
R¹ and R² are each independently H, OH or alkoxy;
R³ is alkyl, aryl or heterocyclyl;
R⁴ is alkyl or aryl;
R⁵ is aryl;
X¹ is NH, S or O; and
X², X³, and X⁴ are each, independently, alkyl.

An example of a compound of the formula (I) is a compound of the formula (Ia):

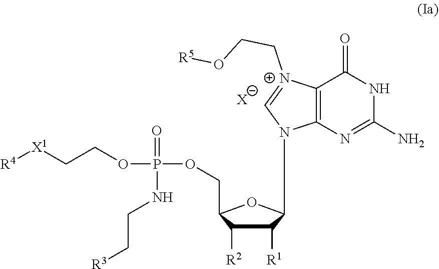

or a pharmaceutically acceptable salt thereof.

The compound of the formula (I) can also be a compound of the formula (II):

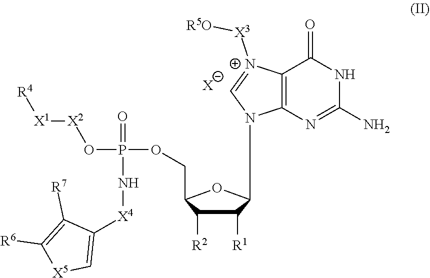

or a pharmaceutically acceptable salt thereof;
wherein:
X⁻ is a counterion, such as chloride and the like;
R¹ and R² are each independently H, OH or alkoxy;
R⁴ is alkyl or aryl;
R⁵ is aryl;
X¹ and X⁵ are each independently NH, S or O;
X², X³, and X⁴ are each, independently, alky; and
R⁶ and R⁷ can each independently be H, alkyl, aryl or R⁶ and R⁷, together with the carbon atoms to which they are attached, can form an aryl group.

An example of a compound of the formula (II) is a compound of the formula (IIa):

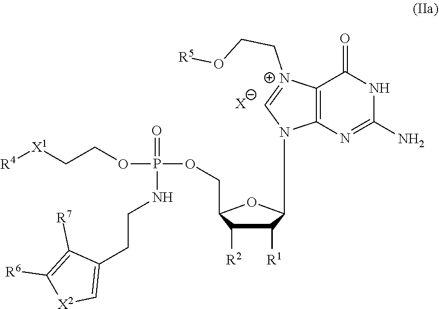

or a pharmaceutically acceptable salt thereof.

In the compounds of the formulae (I), (II), and (IIa), at least one of R¹ and R² is hydroxy and, in some instances, both R¹ and R² can be hydroxy. In addition, or alternatively, or in addition, R³ can be a (C₃-C₇)heterocyclyl group, such as a (C₃-C₇)heteroaryl group, such as a heterocyclyl group of the formula:

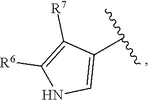

wherein R⁶ and R⁷ can each independently be H, alkyl, aryl or R⁶ and R⁷, together with the carbon atoms to which they are attached, can form an aryl group. For example, R⁶ and R⁷, together with the carbon atoms to which they are attached, can form an aryl group, such as a group of the formula:

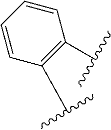 [SMILES: *c1ccccc1*]

that can be optionally substituted. Alternatively or in addition, R⁴ is alkyl, such as (C₁-C₃)alkyl. Alternatively or in addition, X¹ can be S. Alternatively or in addition. R⁵ is (C₆-C₁₀)aryl, such as phenyl or napthyl each of which can be optionally substituted, e.g., with halo, such as chloro. Alternatively or in addition, X², X³, and X⁴ are each, independently (C₁-C₆)alkyl, such as (C₁-C₃)alkyl. In some examples, X³ and X⁴ are each, independently, (C₁-C₃)alkyl. Alternatively or in addition, X² is (C₁-C₅)alkyl, such as (C₂-C₅)alkyl.

Compounds that fall within the scope of the formulae (I), (Ia), (II), and (IIa) are the compounds of formulae (III) and (IV) or a pharmaceutically acceptable salt thereof:

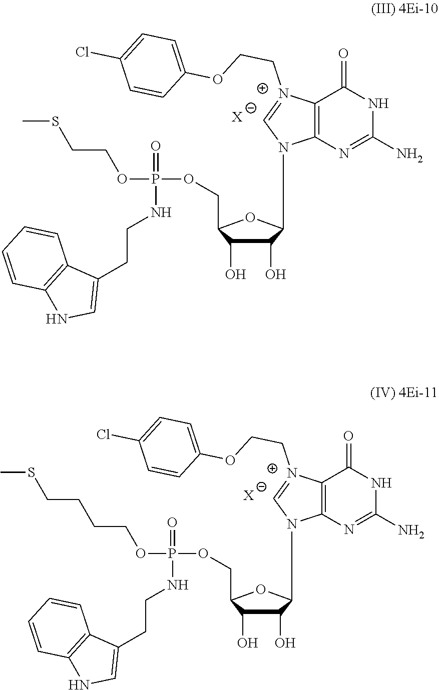

wherein:
X⁻ is a counterion, such as chloride and the like.

The compounds of formula (I), (Ia), (II), (IIa), (III) and (IV), or a pharmaceutically acceptable salt thereof, can be used generally in methods for treating a severe acute respiratory syndrome, the methods comprising administering a therapeutically effective amount of at least one compound of formula (I), (Ia), (II), (IIa), (III) and (IV), or a pharmaceutically acceptable salt thereof, to a subject, such as a mammal (e.g., a human), in need thereof.

As used herein, the term “severe acute respiratory syndrome” includes, but is not limited to, a viral respiratory disease caused by a SARS-associated coronavirus and variants thereof. An example of a coronavirus is the so-called novel coronavirus, which is also known as COVID-19 and SARS-CoV-2. Variants of COVID-19 include the so-called delta variant and the so-called omicron variant.

In the aforementioned method, the at least one compound of formula (I), (Ia), (II), (IIa), (III) and (IV), or a pharmaceutically acceptable salt thereof, can be administered either alone or in combination with another antiviral agent. For example, the at least one compound of formula (I), (Ia), (II), (IIa), (III) and (IV), or a pharmaceutically acceptable salt thereof, can be administered in combination with a small molecule for treating COVID-19. Examples, of such small molecules include, but are not limited to, remdesivir, paxlovid, and molnupiravir.

The term “alkyl” as used herein refers to substituted or unsubstituted straight chain, branched and cyclic, saturated mono-valent groups having from 1 to 20 carbon atoms, 10 to 20 carbon atoms, 12 to 18 carbon atoms, 6 to about 10 carbon atoms, 1 to 10 carbons atoms, 1 to 8 carbon atoms, 2 to 8 carbon atoms, 3 to 8 carbon atoms, 4 to 8 carbon atoms, 5 to 8 carbon atoms, 1 to 6 carbon atoms, 2 to 6 carbon atoms, 3 to 6 carbon atoms, or 1 to 3 carbon atoms. Examples of straight chain mono-valent (C₁-C₂₀)-alkyl groups include those with from 1 to 8 carbon atoms such as methyl (i.e., CH₃), ethyl, n-propyl, n-butyl, n-pentyl, n-hexyl, n-heptyl, n-octyl groups. Examples of branched mono-valent (C₁-C₂₀)-alkyl groups include isopropyl, iso-butyl, sec-butyl, t-butyl, neopentyl, and isopentyl. Examples of cyclic alkyl groups include cyclopropyl, cyclobutyl, cyclopently, cyclohexyl, cyclooctyl, bicyclo[1.1.1]pentyl, bicyclo[2.1.1]hexyl, and bicyclo[2.2.1]heptyl. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Alkyl includes a combination of substituted and unsubstituted alkyl. As an example, alkyl, and also (C₁)alkyl, includes methyl and substituted methyl. As a particular example, (C₁)alkyl includes benzyl. As a further example, alkyl can include methyl and substituted (C₂-C₈)alkyl. Alkyl can also include substituted methyl and unsubstituted (C₂-C₈)alkyl. Alkyl can be methyl and C₂-C₈ linear alkyl. Alkyl can be methyl and C₂-C₈ branched alkyl. The term methyl is understood to be —CH₃, which is not substituted. For comparison, the term (C₁)alkyl is understood to be a substituted or an unsubstituted —CH₃. Representative substituted alkyl groups can be substituted one or more times with any of the groups listed herein, for example, cycloalkyl, heterocyclyl, aryl, amino, haloalkyl, hydroxy, cyano, carboxy, nitro, thio, alkoxy, and halogen groups. As further example, representative substituted alkyl groups can be substituted one or more fluoro, chloro, bromo, iodo, amino, amido, alkyl, alkoxy, alkylamido, alkenyl, alkynyl, alkoxycarbonyl, acyl, formyl, arylcarbonyl, aryloxycarbonyl, aryloxy, carboxy, haloalkyl, hydroxy, cyano, nitroso, nitro, azido, trifluoromethyl, trifluoromethoxy, thio, alkylthio, arylthiol, alkylsulfonyl, alkylsulfinyl, dialkylaminosulfonyl, sulfonic acid, carboxylic acid, dialkylamino and dialkylamido. The term “alkyl” also generally refers to alkyl groups that can comprise one or more heteroatoms in the carbon chain. Thus, for example, “alkyl” also encompasses groups such as —[(CH₂)ₚO]_{q}H and the like.

The term “aryl” as used herein refers to substituted or unsubstituted univalent groups that are derived by removing a hydrogen atom from an arene, which is a cyclic aromatic hydrocarbon, having from 6 to 20 carbon atoms, 10 to 20 carbon atoms, 12 to 20 carbon atoms, 6 to about 10 carbon atoms or 6 to 8 carbon atoms. Examples of (C₆-C₂₀)aryl groups include phenyl, napthalenyl, azulenyl, biphenylyl, indacenyl, fluorenyl, phenanthrenyl, triphenylenyl, pyrenyl, naphthacenyl, chrysenyl, anthracenyl groups. Examples include substituted phenyl, substituted napthalenyl, substituted azulenyl, substituted biphenylyl, substituted indacenyl, substituted fluorenyl, substituted phenanthrenyl, substituted triphenylenyl, substituted pyrenyl, substituted naphthacenyl, substituted chrysenyl, and substituted anthracenyl groups. Examples also include unsubstituted phenyl, unsubstituted napthalenyl, unsubstituted azulenyl, unsubstituted biphenylyl, unsubstituted indacenyl, unsubstituted fluorenyl, unsubstituted phenanthrenyl, unsubstituted triphenylenyl, unsubstituted pyrenyl, unsubstituted naphthacenyl, unsubstituted chrysenyl, and unsubstituted anthracenyl groups. From these examples, it is clear that the term (C₆-C₂₀)aryl encompasses mono-and polycyclic (C₆-C₂₀)aryl groups, including fused and non-fused polycyclic (C₆-C₂₀)aryl groups.

The term “heterocyclyl” as used herein refers to substituted aromatic, unsubstituted aromatic, substituted non-aromatic, and unsubstituted non-aromatic rings containing 3 or more atoms in the ring, of which, one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. In some embodiments, heterocyclyl groups include heterocyclyl groups that include 3 to 8 carbon atoms (C₃-C₈), 3 to 6 carbon atoms (C₃-C₆) or 6 to 8 carbon atoms (C₆-C₈). A heterocyclyl group designated as a C₂-heterocyclyl can be a 5-membered ring with two carbon atoms and three heteroatoms, a 6-membered ring with two carbon atoms and four heteroatoms and so forth. Likewise a C₄-heterocyclyl can be a 5-membered ring with one heteroatom, a 6-membered ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. Representative heterocyclyl groups include, but are not limited to piperidynyl, piperazinyl, morpholinyl, furanyl, pyrrolidinyl, pyridinyl, pyrazinyl, pyrimidinyl, triazinyl, thiophenyl, tetrahydrofuranyl, pyrrolyl, oxazolyl, imidazolyl, triazyolyl, tetrazolyl, indolyl, benzoxazolinyl, and benzimidazolinyl groups. For example, heterocyclyl groups include, without limitation:

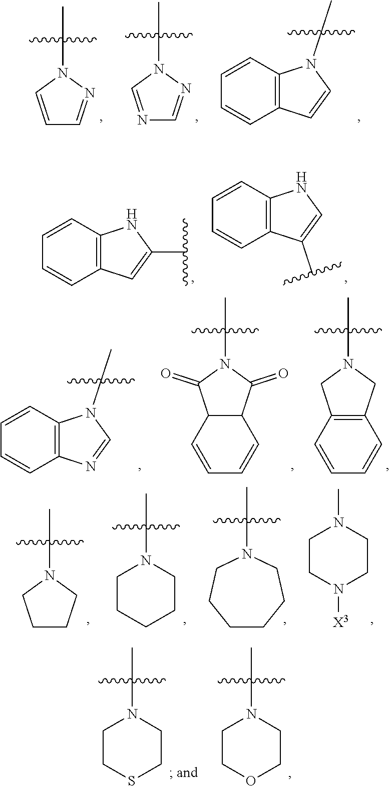

wherein X³ represents H, (C₁-C₂₀)alkyl, (C₆-C₂₀)aryl or an amine protecting group (e.g., a t-butyloxycarbonyl group) and wherein the heterocyclyl group can be substituted or unsubstituted. A nitrogen-containing heterocyclyl group is a heterocyclyl group containing a nitrogen atom as an atom in the ring. In some embodiments, the heterocyclyl is other than thiophene or substituted thiophene. In some embodiments, the heterocyclyl is other than furan or substituted furan.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include one to about 12-20 or about 12-40 carbon atoms bonded to the oxygen atom, and can further include double or triple bonds, and can also include heteroatoms. Thus, alkyoxy also includes an oxygen atom connected to an alkyenyl group and oxygen atom connected to an alkynyl group. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

The terms “halo,” “halogen,” or “halide” group, as used herein, by themselves or as part of another substituent, mean, unless otherwise stated, a fluorine, chlorine, bromine, or iodine atom.

The term “substituted” as used herein refers to a group that is substituted with one or more groups including, but not limited to, the following groups: halogen (e.g., F, Cl, Br, and I), R, OR, OC(O)N(R)₂, CN, NO, NO₂, ONO₂, azido, CF₃, OCF₃, methylenedioxy, ethylenedioxy, (C₃-C₂₀)heteroaryl, N(R)₂, Si(R)₃, SR, SOR, SO₂R, SO₂N(R)₂, SO₃R, P(O)(OR)₂, OP(O)(OR)₂, C(O)R, C(O)C(O)R, C(O)CH₂C(O)R, C(S)R, C(O)OR, OC(O)R, C(O)N(R)₂, C(O)N(R)OH, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, or C(═NOR)R wherein R can be hydrogen, (C₁-C₂₀)alkyl or (C₆-C₂₀)aryl. Substituted also includes a group that is substituted with one or more groups including, but not limited to, the following groups: fluoro, chloro, bromo, iodo, amino, amido, alkyl, alkoxy, alkylamido, alkenyl, alkynyl, alkoxycarbonyl, acyl, formyl, arylcarbonyl, aryloxycarbonyl, aryloxy, carboxy, haloalkyl, hydroxy, cyano, nitroso, nitro, azido, trifluoromethyl, trifluoromethoxy, thio, alkylthio, arylthiol, alkylsulfonyl, alkylsulfinyl, dialkylaminosulfonyl, sulfonic acid, carboxylic acid, dialkylamino and dialkylamido. Where there are two or more adjacent substituents, the substituents can be linked to form a carbocyclic or heterocyclic ring. Such adjacent groups can have a vicinal or germinal relationship, or they can be adjacent on a ring in, e.g., an ortho-arrangement. Each instance of substituted is understood to be independent. For example, a substituted aryl can be substituted with bromo and a substituted heterocycle on the same compound can be substituted with alkyl. It is envisaged that a substituted group can be substituted with one or more non-fluoro groups. As another example, a substituted group can be substituted with one or more non-cyano groups. As another example, a substituted group can be substituted with one or more groups other than haloalkyl. As yet another example, a substituted group can be substituted with one or more groups other than tert-butyl. As yet a further example, a substituted group can be substituted with one or more groups other than trifluoromethyl. As yet even further examples, a substituted group can be substituted with one or more groups other than nitro, other than methyl, other than methoxymethyl, other than dialkylaminosulfonyl, other than bromo, other than chloro, other than amido, other than halo, other than benzodioxepinyl, other than polycyclic heterocyclyl, other than polycyclic substituted aryl, other than methoxycarbonyl, other than alkoxycarbonyl, other than thiophenyl, or other than nitrophenyl, or groups meeting a combination of such descriptions. Further, substituted is also understood to include fluoro, cyano, haloalkyl, tert-butyl, trifluoromethyl, nitro, methyl, methoxymethyl, dialkylaminosulfonyl, bromo, chloro, amido, halo, benzodioxepinyl, polycyclic heterocyclyl, polycyclic substituted aryl, methoxycarbonyl, alkoxycarbonyl, thiophenyl, and nitrophenyl groups.

This disclosure also contemplates pharmaceutical compositions comprising the at least one compound of formula (I), (Ia), (II), (IIa), (III) and (IV), or a pharmaceutically acceptable salt thereof, and one or more pharmaceutically acceptable carriers, diluents, excipients or combinations thereof. A “pharmaceutical composition” refers to a chemical or biological composition suitable for administration to a subject (e.g., mammal). Such compositions may be specifically formulated for administration via one or more of a number of routes, including but not limited to buccal, cutaneous, epicutaneous, epidural, infusion, inhalation, intraarterial, intracardial, intracerebroventricular, intradermal, intramuscular, intranasal, intraocular, intraperitoneal, intraspinal, intrathecal, intravenous, oral, parenteral, pulmonary, rectally via an enema or suppository, subcutaneous, subdermal, sublingual, transdermal, and transmucosal. In addition, administration can by means of capsule, drops, foams, gel, gum, injection, liquid, patch, pill, porous pouch, powder, tablet, or other suitable means of administration.

A “pharmaceutical excipient” or a “pharmaceutically acceptable excipient” comprises a carrier, sometimes a liquid, in which an active therapeutic agent is formulated. The excipient generally does not provide any pharmacological activity to the formulation, though it may provide chemical and/or biological stability, and release characteristics. Examples of suitable formulations can be found, for example, in Remington, The Science And Practice of Pharmacy, 20th Edition, (Gennaro, A. R., Chief Editor), Philadelphia College of Pharmacy and Science, 2000, which is incorporated by reference in its entirety.

As used herein “pharmaceutically acceptable carrier” or “excipient” includes any and all solvents, dispersion media, coatings, antibacterial and antifungal agents, isotonic and absorption delaying agents that are physiologically compatible. In one example, the carrier is suitable for parenteral administration. Alternatively, the carrier can be suitable for intravenous, intraperitoneal, intramuscular, sublingual, or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Pharmaceutical compositions may be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (e.g., glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants.

In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, the compounds described herein can be formulated in a time release formulation, for example in a composition that includes a slow release polymer. The active compounds can be prepared with carriers that will protect the compound against rapid release, such as a controlled release formulation, including implants and microencapsulated delivery systems. Biodegradable, biocompatible polymers may be used, such as ethylene vinyl acetate, polyanhydrides, polyglycolic acid, collagen, polyorthoesters, polylactic acid and polylactic, polyglycolic copolymers (PLG). Many methods for the preparation of such formulations are known to those skilled in the art.

Oral forms of administration are also contemplated herein. The pharmaceutical compositions of the present invention may be orally administered as a capsule (hard or soft), tablet (film coated, enteric coated or uncoated), powder or granules (coated or uncoated) or liquid (solution or suspension). The formulations may be conveniently prepared by any of the methods well-known in the art. The pharmaceutical compositions of the present invention may include one or more suitable production aids or excipients including fillers, binders, disintegrants, lubricants, diluents, flow agents, buffering agents, moistening agents, preservatives, colorants, sweeteners, flavors, and pharmaceutically compatible carriers.

For each of the recited examples, the compounds can be administered by a variety of dosage forms as known in the art. Any biologically-acceptable dosage form known to persons of ordinary skill in the art, and combinations thereof, are contemplated. Examples of such dosage forms include, without limitation, chewable tablets, quick dissolve tablets, effervescent tablets, reconstitutable powders, elixirs, liquids, solutions, suspensions, emulsions, tablets, multi-layer tablets, bi-layer tablets, capsules, soft gelatin capsules, hard gelatin capsules, caplets, lozenges, chewable lozenges, beads, powders, gum, granules, particles, microparticles, dispersible granules, cachets, douches, suppositories, creams, topicals, inhalants, aerosol inhalants, patches, particle inhalants, implants, depot implants, ingestibles, injectables (including subcutaneous, intramuscular, intravenous, and intradermal), infusions, and combinations thereof.

Other compounds which can be included by admixture are, for example, medically inert ingredients (e.g., solid and liquid diluent), such as lactose, dextrosesaccharose, cellulose, starch or calcium phosphate for tablets or capsules, olive oil or ethyl oleate for soft capsules and water or vegetable oil for suspensions or emulsions; lubricating agents such as silica, talc, stearic acid, magnesium or calcium stearate and/or polyethylene glycols; gelling agents such as colloidal clays; thickening agents such as gum tragacanth or sodium alginate, binding agents such as starches, arabic gums, gelatin, methylcellulose, carboxymethylcellulose or polyvinylpyrrolidone; disintegrating agents such as starch, alginic acid, alginates or sodium starch glycolate; effervescing mixtures; dyestuff; sweeteners; wetting agents such as lecithin, polysorbates or laurylsulphates; and other therapeutically acceptable accessory ingredients, such as humectants, preservatives, buffers and antioxidants, which are known additives for such formulations.

Liquid dispersions for oral administration can be syrups, emulsions, solutions, or suspensions. The syrups can contain as a carrier, for example, saccharose or saccharose with glycerol and/or mannitol and/or sorbitol. The suspensions and the emulsions can contain a carrier, for example a natural gum, agar, sodium alginate, pectin, methylcellulose, carboxymethylcellulose, or polyvinyl alcohol.

The amount of active compound in a therapeutic composition according to various examples of the present invention may vary according to factors such as the disease state, age, gender, weight, patient history, risk factors, predisposition to disease, administration route, pre-existing treatment regime (e.g., possible interactions with other medications), and weight of the individual. Dosage regimens may be adjusted to provide the optimum therapeutic response. For example, a single bolus may be administered, several divided doses may be administered over time, or the dose may be proportionally reduced or increased as indicated by the exigencies of therapeutic situation.

“Dosage unit form,” as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of sensitivity in individuals. In therapeutic use for treatment of conditions in mammals (e.g., humans) for which the compounds of the present invention or an appropriate pharmaceutical composition thereof are effective, the compounds of the present invention may be administered in an effective amount. The dosages as suitable for this invention may be a composition, a pharmaceutical composition or any other compositions described herein.

For each of the recited examples, the dosage is typically administered once, twice, or thrice a day, although more frequent dosing intervals are possible. The dosage may be administered every day, every 2 days, every 3 days, every 4 days, every 5 days, every 6 days, and/or every 7 days (once a week). In one example, the dosage may be administered daily for up to and including 30 days, preferably between 7-10 days. In another example, the dosage may be administered twice a day for 10 days. If the patient requires treatment for a chronic disease or condition, the dosage may be administered for as long as signs and/or symptoms persist. The patient may require “maintenance treatment” where the patient is receiving dosages every day for months, years, or the remainder of their lives. In addition, the composition of this invention may be to effect prophylaxis of recurring symptoms. For example, the dosage may be administered once or twice a day to prevent the onset of symptoms in patients at risk, especially for asymptomatic patients.

The compositions described herein may be administered in any of the following routes: buccal, epicutaneous, epidural, infusion, inhalation, intraarterial, intracardial, intracerebroventricular, intradermal, intramuscular, intranasal, intraocular, intraperitoneal, intraspinal, intrathecal, intravenous, oral, parenteral, pulmonary, rectally via an enema or suppository, subcutaneous, subdermal, sublingual, transdermal, and transmucosal. The preferred routes of administration are buccal and oral. The administration can be local, where the composition is administered directly, close to, in the locality, near, at, about, or in the vicinity of, the site(s) of disease, e.g., inflammation, or systemic, wherein the composition is given to the patient and passes through the body widely, thereby reaching the site(s) of disease. Local administration can be administration to the cell, tissue, organ, and/or organ system, which encompasses and/or is affected by the disease, and/or where the disease signs and/or symptoms are active or are likely to occur. Administration can be topical with a local effect, composition is applied directly where its action is desired. Administration can be enteral wherein the desired effect is systemic (non-local), composition is given via the digestive tract. Administration can be parenteral, where the desired effect is systemic, composition is given by other routes than the digestive tract.

This disclosure also contemplates pharmaceutical compositions comprising at least one compound of formula (I), (Ia), (II), (IIa), (III) and (IV), or a pharmaceutically acceptable salt thereof, for use as a medicament for treating a patient in need of relief from a severe acute respiratory syndrome.

The term “therapeutically effective amount” as used herein, refers to that amount of one or more compounds of the various examples of the present invention that elicits a biological or medicinal response in a tissue system, animal or human, that is being sought by a researcher, veterinarian, medical doctor or other clinician, which includes alleviation of the symptoms of the disease or disorder being treated. In some examples, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the condition being treated and the severity of the condition; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician. It is also appreciated that the therapeutically effective amount can be selected with reference to any toxicity, or other undesirable side effect, that might occur during administration of one or more of the compounds described herein.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include

one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference.

In the methods described herein, the steps can be carried out in any order without departing from the principles of the invention, except when a temporal or operational sequence is explicitly recited. Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range.

As used herein, the term “salts” and “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound which contains a basic or acidic moiety by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, the disclosure of which is hereby incorporated by reference.

The disclosure also contemplates solvates and prodrugs of at least one compound of formula (I), (Ia), (II), (IIa), (III) and (IV).

The term “solvate” means a compound, or a salt thereof, that further includes a stoichiometric or non-stoichiometric amount of solvent bound by non-covalent intermolecular forces. Where the solvent is water, the solvate is a hydrate.

The term “prodrug” means a derivative of a compound that can hydrolyze, oxidize, or otherwise react under biological conditions (in vitro or in vivo) to provide an active compound, particularly a compound of the invention. Examples of prodrugs include, but are not limited to, derivatives and metabolites of a compound of the invention that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Specific prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6th ed. (Donald J. Abraham ed., 2001, Wiley) and Design and Application of Prodrugs (H. Bundgaard ed., 1985, Harwood Academic Publishers GmbH).

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present disclosure. Thus, it should be understood that although the present disclosure has been specifically disclosed by specific embodiments and optional features, modification and variation of the concepts herein disclosed can be resorted to by those of ordinary skill in the art, and that such modifications and variations are considered to be within the scope of embodiments of the present disclosure

The invention is now described with reference to the following Examples. The following working examples therefore, are provided for the purpose of illustration only and specifically point out certain embodiments of the present invention, and are not to be construed as limiting in any way the remainder of the disclosure. Therefore, the examples should be construed to encompass any and all variations which become evident as a result of the teaching provided herein.

### EXAMPLES

The present invention can be better understood by reference to the following examples which are offered by way of illustration. The present invention is not limited to the examples given herein.

### Example 1: Screening for Antiviral Activity in Vero E6 Cells

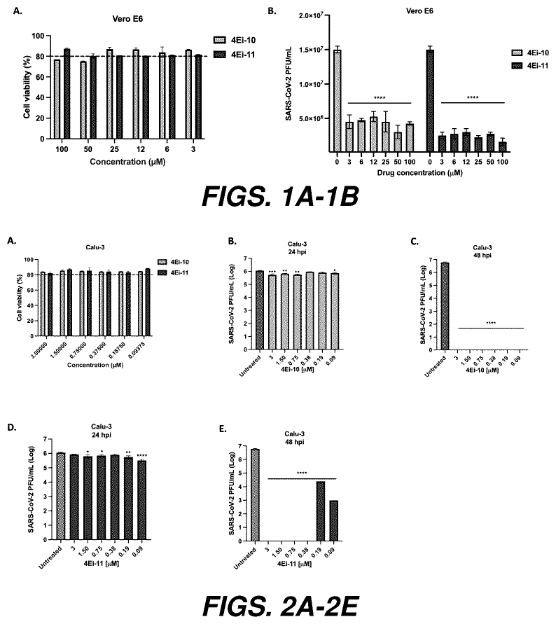

The cellular toxicity of the compounds 4Ei-10 and 4Ei-11 was assessed by CellTiter Aqueous One cell viability assay available from Fisher Scientific and other similar suppliers. Vero E6 cells were treated with each compound at varying concentrations from 3 μM to 100 μM for 48 hours. The data are presented in FIG. 1A.

SARS-CoV-2 (A2 lineage) infectious particles in culture supernatant were titrated to quantify the plaques formed in cell culture upon infection with serial dilutions of virus specimen. Cells were pretreated for 4 hours with each compound at different concentrations (from 3 μM to 100 μM) followed by infection (MOI 0.1). After infection, drugs were replenished in the culture media. Cells were incubated for 48 hours before virus titration. ****p<0.0001 by One-way ANOVA. N=3. The data are presented in FIG. 1B.

### Example 2: Screening for Antiviral Activity in Calu-3 Cells

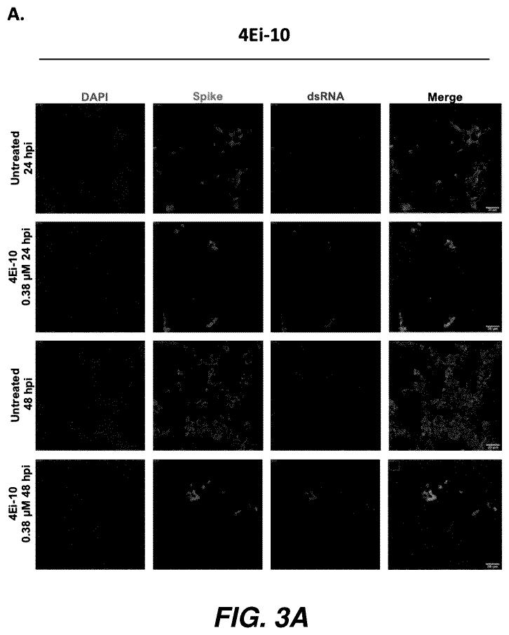

The cellular toxicity of the compounds 4Ei-10 and 4Ei-11 was assessed by CellTiter Aqueous One cell viability assay available from Fisher Scientific and other similar suppliers. Calu-3 cells were treated with each compound at varying concentrations from 0.09 μM to 3 μM for 48 hours. The data are presented in FIG. 2A.

SARS-CoV-2 (A2 lineage) infectious particles in culture supernatant were titrated to quantify the plaques formed in cell culture upon infection with serial dilutions of virus specimen. Cells were pretreated for 4 hours with each compound at different concentrations (from 0.09 μM to 3 μM) followed by infection (MOI 0.1). After infection, drugs were replenished in the culture media. Cells were incubated for 24 or 48 hours before virus titration. *p<0.05; **p<0.01; ***p<0.001; ****p<0.0001 by One-way ANOVA. N=3. The data are presented in FIG. 2B.

### Example 3

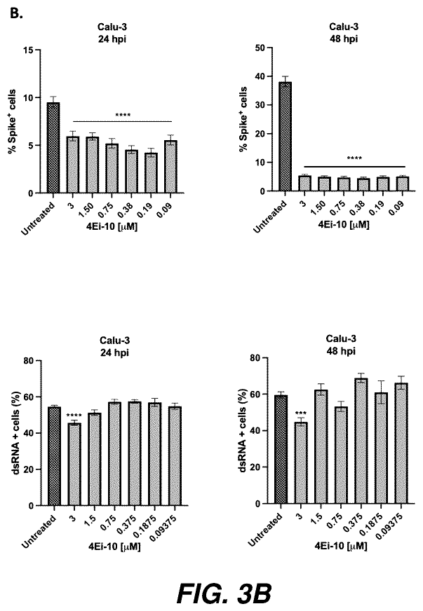

Confocal microscopy was used to visualize SARS-CoV-2 Spike protein and dsRNA in Calu-3 cells (FIG. 3A) treated with the compound 4Ei-10. Cells were pretreated with each compound at 0.38 μM for 4 hours followed by infection with SARS-CoV-2 (Lineage A) at an MOI of 0.1. After 1 hour of virus adsorption, the inoculum was removed and cell media containing the compounds was replenished. Cells were cultured up to 48 hours. After that, supernatants were harvested, and cells were fixed with 4% paraformaldehyde (PFA) and labeled with specific antibodies against viral Spike protein (green) and dsRNA (red). Cells were observed using an ImageXpress Ultra Confocal Microscopy and the images were analyzed by Software MetaXpress version 4.0.0.42 for quantification of puncta. FIG. 3B shows quantification of the images acquired by confocal microscopy. ***p<0.001; ****p<0.0001 by One-way ANOVA.

### Example 4

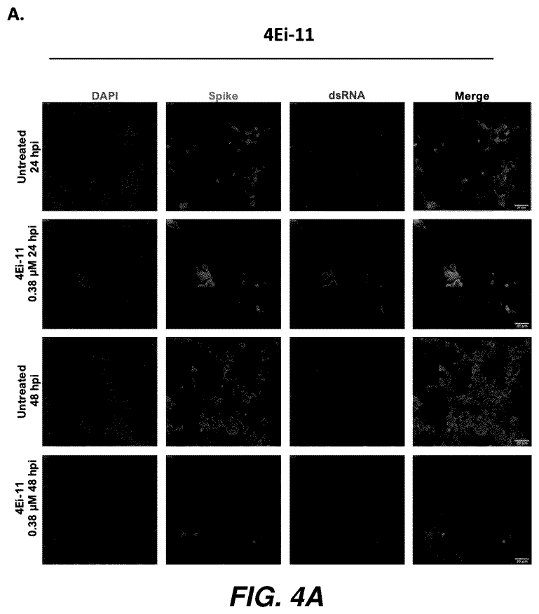

Confocal microscopy was used to visualize of SARS-CoV-2 Spike protein and dsRNA in Calu-3 cells (FIG. 4A) treated with the compound 4Ei-11. Cells were pretreated with each compound at 0.38 μM for 4 hours followed by infection with SARS-CoV-2 (Lineage A) at an MOI of 0.1. After 1 h of virus adsorption, the inoculum was removed and cell media containing the compounds was replenished. Cells were cultured up to 48 hours. After that, supernatants were harvested, and cells were fixed with 4% PFA and labeled with specific antibodies against viral Spike protein (green) and dsRNA (red). Cells were observed using an ImageXpress Ultra Confocal Microscopy and the images were analyzed by Software MetaXpress version 4.0.0.42 for quantification of puncta. FIG. 4B shows 1qantification of the images acquired by confocal microscopy. *p<0.05; ***p<0.001; ****p<0.0001 by One-way ANOVA.

### Example 5

Antiviral activity of the compounds 4Ei-10 and 4Ei-11 against SARS-CoV-2 (Omicron VOC) was evaluated.

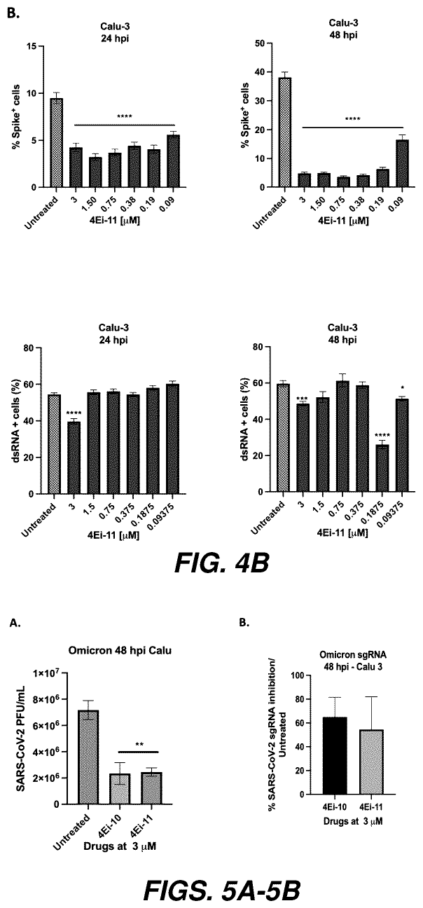

SARS-CoV-2 (A2 lineage) infectious particles in culture supernatant were titrated to quantify the plaques formed in cell culture upon infection with serial dilutions of virus specimen. Cells were pretreated for 4 hours with each compound at different concentrations (from 3 μM to 100 μM) followed by infection (MOI 0.1). After infection, drugs were replenished in the culture media. Cells were incubated for 48 hours before virus titration. **p<0.01 by One-way ANOVA. N=3. The data are shown in FIG. 5A. FIG. 5B shows the percentage of inhibition of the amount of viral dsRNA produced. Cells were pretreated for 4 hours with each compound at 3 μM to 100 μM followed by infection (MOI 0.1). After infection, drugs were replenished in the culture media. Cells were cultured for 48 hours. After that, cell lysate was processed for RNA extraction followed by cDNA synthesis and qPCR with specific primers to dsRNA. The percentage of dsRNA was quantified by ΔΔCt method and normalized by the untreated control condition.

What is claimed is:

1. A method for treating an influenza viral infection or a severe acute respiratory syndrome, the method comprising administering a therapeutically effective amount of at least one compound of the formula (I): 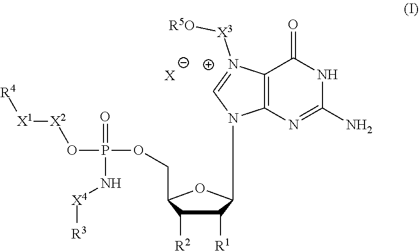
or a pharmaceutically acceptable salt thereof,
wherein:
X⁻ is a counterion, such as chloride;
R¹ and R² are each independently H, OH or alkoxy;
R³ is alkyl, aryl or heterocyclyl;
R⁴ is alkyl or aryl;
R⁵ is aryl;
X¹ is NH, S or O; and
X², X³, and X⁴ are each, independently, alkyl;
or a pharmaceutical composition comprising a therapeutically effective amount of one or more compounds of the formula (I), whereupon the patient is treated for a severe acute respiratory syndrome.

2. The method of claim 1, wherein the severe acute respiratory syndrome is COVID-19.

3. The method of claim 1, wherein the influenza viral infection is caused by an influenza A virus.

4. The method of claim 1, wherein the influenza virus A is subtype H1N1, H2N2, H3N2, or H5N1.

5. The method of claim 1, wherein the influenza viral infection is caused by an influenza B virus.

6. The method of claim 1, wherein the at least one compound of the formula (I) is a compound of the formula (Ia): 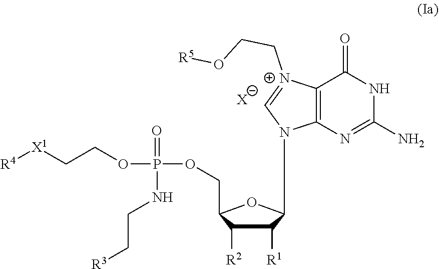
or a pharmaceutically acceptable salt thereof.

7. The method of claim 1, wherein the at least one compound of the formula (I) is a compound of the formula (II): 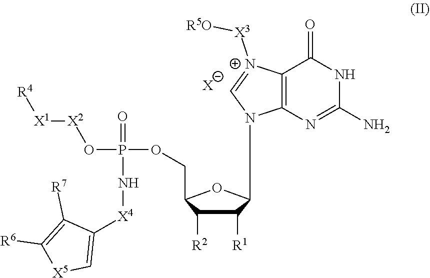
or a pharmaceutically acceptable salt thereof;
wherein:
X⁻ is a counterion, such as chloride;
R¹ and R² are each independently H, OH or alkoxy;
R⁴ is alkyl or aryl;
R⁵ is aryl;
X¹ and X⁵ are each independently NH, S or O;
X², X³, and X⁴ are each, independently, alky; and
R⁶ and R⁷ can each independently be H, alkyl, aryl or R⁶ and R⁷, together with the carbon atoms to which they are attached, can form an aryl group.

8. The method of claim 1, wherein the compound of the formula (II) is a compound of the formula (IIa): 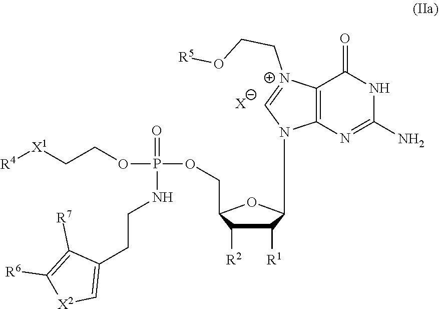
or a pharmaceutically acceptable salt thereof.

9. The method of claim 1, wherein at least one of R¹ and R² is hydroxy.

10. The method of claim 9, wherein R¹ and R² are hydroxy.

11. The method of claim 1, wherein R³ is a (C₃-C₇)heterocyclyl group.

12. The method of claim 11, wherein R³ is (C₃-C₇)heteroaryl group.

13. The method of claim 11, wherein R³ is a heterocyclyl group of the formula: 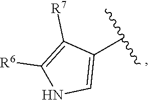
wherein R⁶ and R⁷ is each independently H, alkyl, aryl or R⁶ and R⁷, together with the carbon atoms to which they are attached, form an aryl group.

14. The method of claim 13, wherein R⁶ and R⁷, together with the carbon atoms to which they are attached, form an aryl group of the formula:

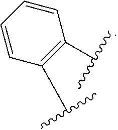 [SMILES: *c1ccccc1*]

15. The method of claim 1, wherein R⁴ is alkyl.

16. The method of claim 15, wherein R⁴ is (C₁-C₃)alkyl.

17. The method of claim 1, wherein X¹ is S.

18. The method of claim 1, wherein R⁵ is (C₆-C₁₀)aryl.

19. The method of claim 18, wherein R⁵ is phenyl.

20. The method of claim 18, wherein R⁵ is phenyl substituted with halo.

21. The method of claim 20, wherein halo is chloro.

22. The method of claim 1, wherein X², X³, and X⁴ are each, independently (C₁-C₆)alkyl.

23. The method of claim 22, wherein X³ and X⁴ are each, independently, (C₁-C₃)alkyl.

24. The method of claim 22, wherein X² is (C₁-C₅)alkyl.

25. The method of claim 24, wherein X² is (C₂-C₅)alkyl.

26. The method of claim 1, wherein the compound is a compound of the formula: 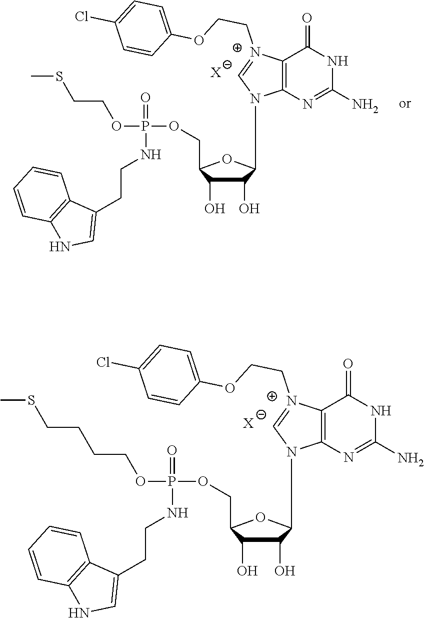
or a pharmaceutically acceptable salt thereof;
wherein:
X⁻ is a counterion, such as chloride.
